(19) United States
(12) Patent Application Publication
(10) Pub. No.: US 2019/0261931 A1
(43) Pub. Date: Aug. 29, 2019

(54) VIDEO PATIENT TRACKING FOR MEDICAL IMAGING GUIDANCE

(71) Applicant: Steven Aaron Ross, El Paso, TX (US)

(72) Inventor: Steven Aaron Ross, El Paso, TX (US)

(21) Appl. No.: 16/287,470

(22) Filed: Feb. 27, 2019

Related U.S. Application Data
(60) Provisional application No. 62/636,097, filed on Feb. 27, 2018.

(51) Int. Cl. A61B 6/04; A61B 5/055; A61B 6/00; A61B 6/03

(52) U.S. Cl. CPC A61B 6/0492; A61B 5/0555; A61B 90/361; A61B 6/032; A61B 6/52; A61B 6/547

(57) ABSTRACT

One or more optical video cameras connected to a monitor correlated to the position of a medical imaging device or patient allows the medical imaging device user to properly align the medical imaging device with respect to the patient for optimal patient medical imaging. The positioning device is used to reduce radiation exposure in modalities using ionizing radiation by allowing optical patient tracking rather than radiation dependent visualization. In all modalities that do or do not use ionizing radiation, it can be used to improve initial patient and/or medical imaging device position and allow users a visual feedback tool to correctly position the patient and/or medical imaging device during the exam.

### PRIORITY APPLICATION

This application claims priority to U.S. Provisional Application Ser. No. 62/636,097, filed Feb. 27, 2018, the disclosure of which is incorporated herein in its entirety by reference.

### TECHNICAL FIELD

The systems and methods described herein relate generally to the use of optical cameras for use in patient positioning and documentation for a plurality of medical imaging modalities.

### BACKGROUND

Currently, there are a number of solutions for positioning patients for medical imaging exams. For x-ray and nuclear medicine, the most common solution is visual assessment by the technologist. In x-ray, sometimes a lamp projecting a rectangle of light over the patient is used for patient and medical imaging device positioning. For fluoroscopy, the most common solution is using fluoroscopy to center the region of interest on the image. Some fluoroscopers keep their hand on the patient to estimate patient position. In computed tomography (CT), the most common solution is to acquire a rapid low-dose “scout” image to prepare the scan. In magnetic resonance imaging, “localizer” or rapid large field of view scans are obtained that allow exam planning. In ultrasound, the technologist manually places the transducer onto the site of interest of the patient.

Therefore, some of these solutions attempt to use a visual gestalt, but these solutions fail to meet the needs of the industry because they are frequently inaccurate and result in repeat imaging. Other solutions attempt to pre-image or rapidly scan the patient, but these solutions are similarly unable to meet the needs of the industry because they can involve additional radiation or increase study time. Still other solutions seek to use the medical imaging modality itself to find patient position, but these solutions also fail to meet industry needs because they involve increased patient radiation dose and/or increased patient imaging time. Finally, other solutions seek to use the fluoroscopists' hands to approximate patient position, but these methods are inaccurate and involve increased ionizing radiation exposure to both the patient and the fluoroscopist.

Thus, a significant component of the time and the patient's radiation dose occurs because the doctor has to hunt for a correct position for the imaging machine in relation to the patient. This is because when the imaging starts the image is not centered and the radiologists use fluoroscopy and other techniques to center the image over the site of interest. There is immense interest in reducing the radiation dose applied to the patient by, for example, eliminating the time required to position the patient and the medical imaging device relative to the patient.

It would be desirable to have a positioning device that allows accurate patient positioning in relationship to a medical imaging device that does not require the use of ionizing radiation, placement of hands on the patient to track movement or position, or the use of the medical imaging device itself. Furthermore, it would also be desirable to have a positioning device that allows visualization of the patient to medical imaging device position in real-time from any proximate or remote location. Still further, it would be desirable to have a positioning device capable of documenting the patient to medical imaging device position.

Therefore, there currently exists a need in the industry for a positioning device and associated method that tracks patient to medical imaging device position so as to allow rapid and accurate medical imaging despite variable patient positions and movement.

### SUMMARY

It is desired to provide rapid and accurate medical imaging by using normal optical cameras to center the image before starting to use fluoroscopy or other imaging modalities. These optical cameras also may be used to “follow” the patient as many patients move their position during the examination requiring further doses to center the images. Optical cameras may address these issues for other modalities as well. For example, optical cameras may be used to set up a scan in CT rather than using an x-ray. The techniques described herein use an optical camera for patient positioning and documentation for a variety of medical imaging modalities.

In sample embodiments, an optical video patient tracking device is provided for patient positioning and medical imaging guidance. The positioning device comprises one or more video cameras and a monitor. The video camera or cameras are connected to the medical imaging device or the patient with a wireless or wired connection to the monitor that is visible to the operator of the medical imaging device. The video camera or cameras are correlated to either the medical imaging device position or to the patient position. As used herein, the term “correlated” means that the cameras are in a known position such that the image defines the physical positional relationship of the patient to the medical imaging device. As used herein, the term “imaging device” refers to any medical imaging device including but not limited to x-ray, computed tomography, magnetic resonance imaging, nuclear medicine imaging, ultrasound, and fluoroscopy.

In the sample embodiments, the positioning device may include one or more of the following: crosshairs or central reticle indicating a mid-position on the monitor, field of view markers on the monitor that indicate the expected field of view of the medical imaging device, a recording device that documents patient position during the imaging process, wires connecting the camera or cameras to the monitor, a wireless device connecting the camera or cameras to the monitor, shutters on the camera lens to optionally prevent imaging, a light or other indicator to signal that the cameras are in use and/or recording, a communication device that sends patient and/or imaging device images to a RIS (radiology information system) or PACS (picture archiving and communication system), a communication device with the medical imaging device that creates field of view markers on the video to indicate medical imaging physical scope, a monitor visible to the patient providing position feedback, and a light illuminating the patient for optimal optical visualization. The cameras are positioned relative to the medical imaging device such that the video will correlate with the acquired medical images. Alternately, the cameras may be connected to the patient and the patient's view of or position relative to the medical imaging device may be captured.

Sample embodiments of the medical imaging system described herein include a medical imaging device that obtains medical images of a patient, a medical imaging monitor that displays the medical images, at least one camera that tracks a position of the patient during imaging by the medical imaging device, and a camera monitor that displays the position of the patient as received from the at least one camera. In the sample embodiments, an output of the at least one camera is correlated with the medical images to illustrate a position of the patient relative to the medical imaging device and/or where the medical images are being taken on the patient relative to the medical imaging device. In sample embodiments, the output of the at least one camera and medical images are correlated such that an output of the at least one camera is centered and limited to a corresponding field of view of a medical image. The medical imaging device may be one of a number of imaging modalities including a computed tomography imaging device, a magnetic resonance imaging device, an ultrasound imaging device, a nuclear medicine imaging device, a mobile radiography imaging device, a stationary radiography imaging device, or a fluoroscopy imaging device.

In the sample embodiments, the at least one camera outputs crosshairs or a central reticle indicating a mid-position of a field of view of the at least one camera on the camera monitor and/or outputs field of view markers on the camera monitor that indicate an expected field of view of the medical imaging device. A communication device associated with the medical imaging device also may be provided to create the field of view markers on an output of the at least one camera to indicate physical scope of medical imaging by the medical imaging device. Also, an output of each camera of the at least one camera may be independently centered to the medical image using markers on the camera output. Shutters optionally may be placed over a lens of the at least one camera to optionally prevent imaging by the at least one camera.

Sample embodiments of the medical imaging device may further include a recording device that records patient position during imaging by the medical imaging device. A communication device may also be provided that sends at least one of medical images of the patient and output of the at least one camera to at least one of a radiology information system and a picture archiving and communication system. In further sample embodiments, the medical imaging device, medical imaging monitor, at least one camera, and camera monitor may be integrated into a same physical structure.

The sample embodiments described herein further include methods of imaging a patient by positioning at least one camera with respect to a medical imaging device to obtain views of axes of interest for imaging by the medical imaging device, imaging a patient with the at least one camera relative to the medical imaging device along the axes of interest of the medical imaging device, using images from the at least one camera to adjust a position of at least one of the patient and the medical imaging device until an imaging position of the patient is in a field of view of the medical imaging device, and imaging the patient at the imaging position using the medical imaging device. The methods may further include imaging the patient with the at least one camera during imaging by the medical imaging device, detecting movement of the patient using differences in output images from the at least one camera during the imaging by the medical imaging device, and alerting an operator of the medical imaging device. In the sample embodiments, outputs of the at least one camera are correlated with outputs of the medical imaging device whereby the at least one camera is in a known position relative to the medical imaging device such that output images from the at least one camera defines a physical positional relationship of the patient to the medical imaging device. Also, outputs of the at least one camera may be used to verify that the patient is performing an activity that is to be performed during imaging by the medical imaging device.

The sample embodiments of the positioning device and associated imaging methods are unique when compared with other known medical imaging devices and solutions because they provide real-time patient positional information, require no additional ionizing radiation, can be used to track the patient during the exam, will allow examination parameter adjustments during the exam, can be used to document position to aid with interpretation later, and can be used to prepare the exam without use of ionizing radiation.

The sample embodiments of the positioning device and associated imaging methods are further unique in that they are structurally different from other known medical imaging devices or solutions. More specifically, the sample embodiments use optical imaging cameras for different medical imaging modalities (e.g., ultrasound, x-ray, fluoroscopy and nuclear medicine), correlated markers indicating the center of the examination and borders of the examination, monitors allowing real-time feedback to the medical imaging device operator, and cameras positioned on the patient or medical imaging device providing positional feedback.

Because the patient position may be determined and tracked in real-time using optical imaging cameras, patient to medical imaging device positional information may be obtained without subjecting the patient to medical imaging ionizing radiation doses.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates monitor views of a two camera optical imaging system used in sample embodiments.

FIG. 2 illustrates a configuration for Computed Tomography or Magnetic Resonance Imaging adapted to include an optical imaging system for positioning the patient in sample embodiments.

FIG. 3 illustrates a configuration for ultrasound imaging adapted to include an optical imaging system for positioning the patient in sample embodiments.

FIG. 4 illustrates a configuration for nuclear medicine imaging adapted to include an optical imaging system for positioning the patient in sample embodiments.

FIG. 5 illustrates a configuration for mobile radiography adapted to include an optical imaging system for positioning the patient in sample embodiments.

FIG. 6 illustrates a configuration for stationary radiography adapted to include an optical imaging system for positioning the patient in sample embodiments.

FIG. 7 illustrates a configuration for fluoroscopy adapted to include an optical imaging system for positioning the patient in sample embodiments.

FIG. 8 illustrates an optional camera shutter system in a sample embodiment.

### DETAILED DESCRIPTION

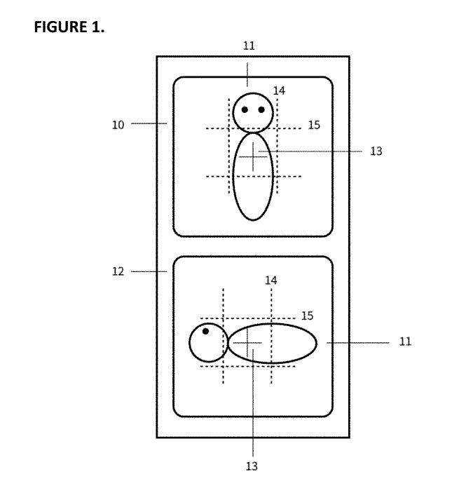

The systems and methods described herein with respect to FIGS. 1-8 are directed to optical video patient tracking for medical imaging. The systems and methods will be described more fully hereinafter with reference to the accompanying drawings, which are intended to be read in conjunction with the summary, the detailed description and any preferred and/or particular embodiments and variations specifically discussed or otherwise disclosed. Embodiments of the system and methods described herein may, however, be embodied in many different forms and should not be construed as limited to the embodiments set forth herein; rather, these embodiments are provided by way of illustration only and so that this disclosure will be thorough, complete and fully convey the full scope of the invention to those skilled in the art.

In sample embodiments, the positioning device includes two or more optical video cameras providing 3-dimensional positional information, a monitor with two or more screen partitions each showing what the cameras are visualizing, a central marker on the screen indicating a central patient or medical imaging device position, boundary markers on the screen indicating the expected boundaries of the medical imaging, camera lens shutters to provide patient privacy when closed, an indicator showing recording and/or active video status of the video cameras, a recording device documenting patient position during imaging, a communication device sending the images to either a RIS (radiology information system) or PACS (picture archiving and communication system), a monitor visible to the patient giving visual feedback on the expected region to be imaged, and a light positioned adjacent to the camera lenses and directed at the patient to illuminate the patient and to provide proper visualization of the patient by the optical cameras in situations where the room or patient location are expected to be dark.

These components are connected and related as follows. The video cameras are connected to either the medical imaging device and/or patient in a correlated way to best visualize the positional relationship of the medical imaging device to the patient. The video cameras are connected wirelessly or in a wired fashion to the monitor which is positioned in a visible location to the medical imaging device user. The shutters are located over the camera lenses and can be changed to either block the lenses or allow visualization through the lenses. The markers on the screens are placed by the optical camera such that the central marker will indicate the central portion of the expected medical image. The boundaries on the screen are placed to indicate the borders of the medical image to be taken. These boundaries will change based on collimation settings on the medical imaging device and other sizing parameters chosen on the medical imaging device. The indicator should be visible to the medical imaging device user and/or the patient. The recording device is also connected to the video cameras in such a way as to allow saving of the acquired videos. The communication device is connected to both the recording device and the local medical imaging system whether it is RIS or PACS. In a sample embodiment, the communication device is implemented as software in the medical imaging device that sends the pictures to the archiving system. Most archiving systems already include such software. The patient monitor is placed in a visible location to the patient so that the patient can get visual feedback on their position and expected medical image.

It should further be noted that the monitors' central and boundary markers will require correlation to the medical imaging device. This correlation includes at least centering of the video cameras such that the optical image has an equivalent center position as the expected medical image. In the simplest form, the correlation would be just equivalent centering; however, lines may be added to the optical image indicating the extent of the fluoroscopic or other medical image. In such a case, the lines would also be correlated to the extent of the fluoroscopic or other medical image. Also, since there can be more than one camera, each camera would be independently centered to the medical image. The boundary markers depend on communication with the medical imaging device to determine the outside margins of the expected medical image. A light illuminating the patient is placed in any location to provide the best optical image.

The medical image and the optical image are correlated such that the optical image of the patient is centered and limited to the corresponding view of the medical image. In sample embodiments, the optical cameras are placed so as not to obstruct the medical image being created; therefore, the cameras cannot be placed within the field of view of the medical image. This creates an offset between the optical camera and the medical image. For this reason, two-dimensional positioning will require two cameras in sample embodiments. The z-axis as defined as the cranial to caudad direction of the patient can be defined by a camera to the side of the medical imaging device imaging the side of the patient. The center of this optical image must correspond to the z-axis center of the medical image. This requires correlation of the optical camera to the medical imaging device by changing the position of the camera until a marker at the center of the medical image is also at the center (z-axis) of the optical image. The x-axis is defined as the left to right direction of the patient and can be defined by a camera to the side of the medical imaging device imaging the top or bottom of the patient. The center of this optical image must correspond to the x-axis center of the medical image. This requires correlation of the optical camera with the medical imaging device by changing the position of the optical camera until a marker at the center of the medical image is also at the center (x-axis) of the optical image. In any modality, a third camera could be correlated in a similar fashion to define the y-axis of the patient (anterior to posterior). The cameras must be positioned such that desired visualization of patient events will be visible by one or more cameras. For example, in fluoroscopy, visualization of patient urination will require the x-axis camera to be placed at the caudad side of the fluoroscopy detector so that the perineum can be visualized during urination.

FIG. 1 illustrates monitor views of a two camera optical imaging system used in sample embodiments. FIG. 1 illustrates the monitors 10 and 12 illustrating the patient 11 and the markings 14 and 15 visible to the user. FIG. 1 illustrates a two-camera system such as would be used in a fluoroscopic setting. Monitor 10 presents the first screen displaying the first camera's view, while monitor 12 presents the second screen displaying the second camera's view. Cross-hairs or a target displaying the center of the optical view 13 are presented as representative of the center of the fluoroscopic image. Markings 14 and 15 represent markers estimating the fluoroscopic image based on the user's collimation setting on the fluoroscopic device.

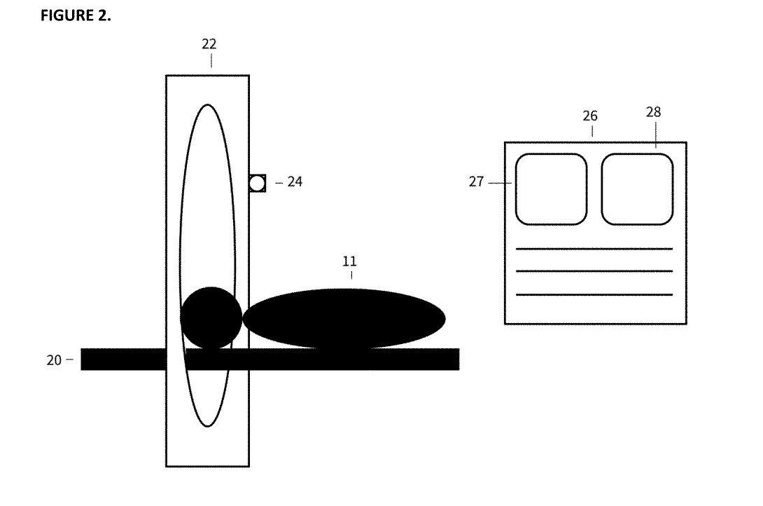

FIG. 2 illustrates a configuration for Computed Tomography or Magnetic Resonance Imaging adapted to include an optical imaging system for positioning the patient in sample embodiments. As illustrated, the patient 11 lies on the CT table 20 and is scanned by the CT scanner and gantry 22. In sample embodiments, the optical camera or cameras 24 are attached to the CT scanner 22 so as to provide views of the patient in one or more planes. Monitor 26 is provided in the technologist's scanning area so as to allow scan planning and verbal instructions to the patient based on position using screens 27 and 28.

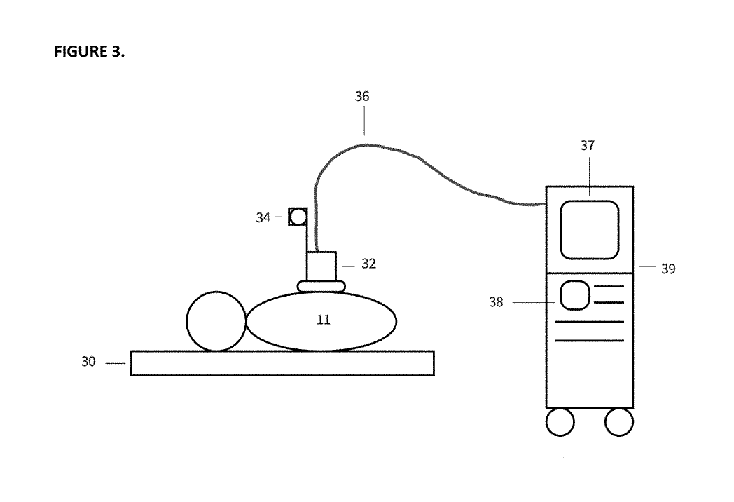

FIG. 3 illustrates a configuration for ultrasound imaging adapted to include an optical imaging system for positioning the patient 11 on the patient's bed 30 in sample embodiments. The transducer 32 is applied to the patient 11 for imaging. Optical camera(s) 34 is/are attached to the transducer 32 or cord 36 in such a way as to document transducer to patient position simultaneous with ultrasound imaging acquisition. The ultrasound monitor 37 provides visualization of the ultrasound image by the sonographer. Monitor 38 shows the optical image obtained by the camera(s) 34 which is also visible to the ultrasonographer. The ultrasound equipment 39 may be modified to include the camera(s) 34 and its monitor 38. For full benefit, the ultrasound equipment 39 may simultaneously record the optical and ultrasound images allowing the radiologist to interpret the ultrasound images while having access to transducer position present during ultrasound acquisition.

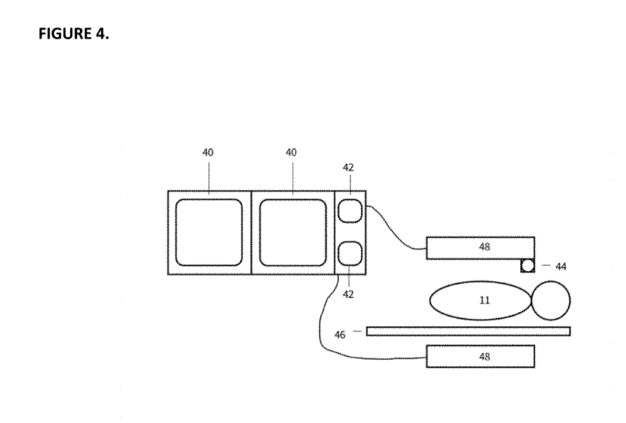

FIG. 4 illustrates a configuration for nuclear medicine imaging adapted to include an optical imaging system for positioning the patient in sample embodiments. As illustrated, nuclear medicine monitors 40 show the acquired nuclear medicine images. Adjacent to monitors 40, monitors 42 are provided to show the optical images of the patient position with respect to the detectors provided by the one or more optical cameras 44. During scanning, the patient 11 lies on bed 46 for the nuclear medicine imaging. Nuclear medicine detectors 48 detect signals that are provided to nuclear medicine equipment for display on the monitors 40.

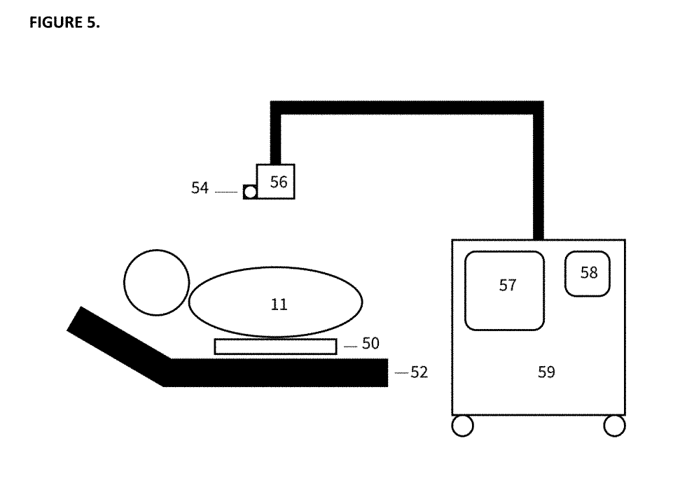

FIG. 5 illustrates a configuration for mobile radiography (x-ray) adapted to include an optical imaging system for positioning the patient 11 in sample embodiments. As illustrated, a detector 50 is placed behind the patient 11 on the patient's bed 52. The optical camera 54 is mounted on the x-ray source 56 to provide an image of the patient. X-ray source 56 provides the x-rays to the patient for creating the x-ray image. The x-ray monitor 57 displays patient information and the acquired x-ray, while monitor 58 displays the optical image of the patient to aid the user in patient positioning. The mobile x-ray unit 59 is modified to incorporate the monitor 58 for positioning the patient during x-ray imaging.

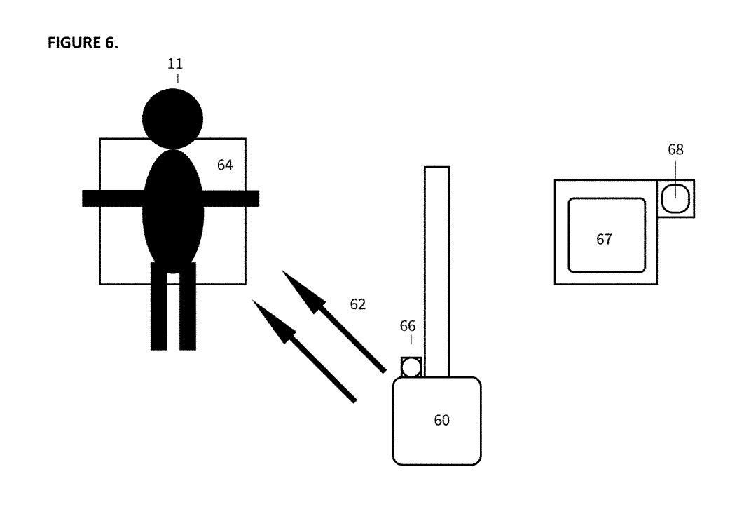

FIG. 6 illustrates a configuration for stationary radiography adapted to include an optical imaging system for positioning the patient in sample embodiments. X-ray source 60 provides x-rays 62 from the x-ray source 60 through the patient 11 to the detector 64 beneath the patient. The optical camera 66 provides images of the patient 11 with respect to the detector 64 and x-ray source 60. The monitor 67 provides information about the patient 11 and the x-ray image to the technologist, while monitor 68 is also visible to the technologist to provide the optical images of the patient position to the technologist.

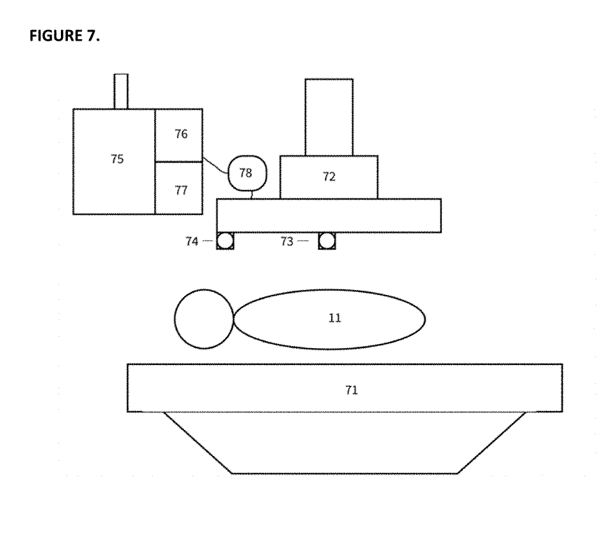

FIG. 7 illustrates a configuration for fluoroscopy adapted to include an optical imaging system for positioning the patient 11 in sample embodiments. The patient 11 is placed on a fluoroscopy table 71 that also houses the fluoroscopy source. In this configuration, the image intensifier 72 receives the x-rays from the fluoroscopy source. One, two or more cameras 73, 74 are affixed to the image intensifier 72 so at to provide patient visualization data to the user with respect to the table 71 and image intensifier 72. Monitor 75 shows the fluoroscopic image to the fluoroscopist, while one, two, or more monitors 76, 77 show the optical images of the patient provided by cameras 73, 74 to the fluoroscopist.

A light may be further provided to illuminate the patient so that the cameras 73, 74 may image an illuminated area to avoid the use of flash. In sample embodiments, the monitor 75 also includes one or more storage devices 78 that record the patient video for later interpretation. In sample embodiments, the monitors 76, 77 are visible to the patient allowing for position feedback to the patient.

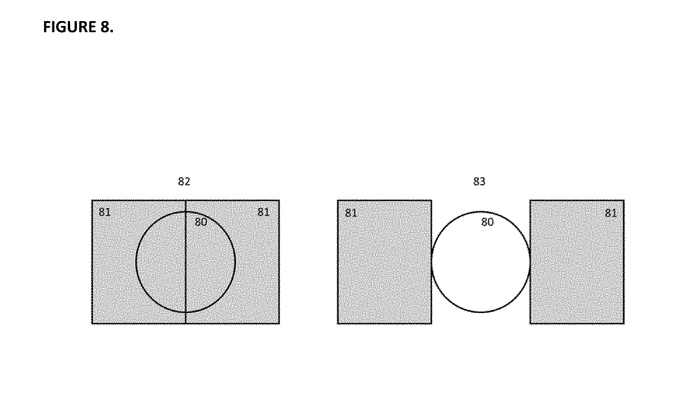

It is noted that many of the optional components of the medical imaging device can be used in this or the other scenarios. For example, a shutter device may be used that blocks the optical camera view when not in use or privacy is desired. FIG. 8 illustrates the optional camera shutter system. On the left-hand side (configuration 82), the shutters 81 are closed preventing use of the optical camera by covering the camera lens 80. On the right-hand side (configuration 83) the shutters 81 are open allowing the optical camera lens 80 to be unobstructed to visualize the patient.

In the respective embodiments, the medical imaging specialist, be it the radiologist or the radiology technologist, will use the optical imaging camera to center the patient in all modalities except ultrasound. Once the patient and medical imaging device are properly centered optically, the markers on the optical image indicating the limits of the medical image are used to also adjust the medical imaging device to only cover the desired portion of the patient. The optical image also may be used to identify patient events such as urination, coughing, etc. during which a medical image is desired allowing for appropriate timing of the image acquisition. On the other hand, for ultrasound, the image of the external patient is obtained simultaneous with the medical image also being sent to the picture archival system allowing an external view of the patient corresponding to the provided internal medical image view.

The prior art methods of image acquisition are modified when using the positioning device as described herein. Without the positioning device the patient position is estimated prior to medical image acquisition; however, with the positioning device, the position of the patient in relationship to the medical imaging device is definitively known by looking at the images from the optical cameras. The axes of interest can be observed by looking at the corresponding camera images. If the patient is offset from the desired position in the x-axis, the patient or medical imaging device can be moved until the correct position is found optically. The medical imaging device is not used until position is confirmed optically. For example, in fluoroscopy without the positioning device, the fluoroscopy machine is used to view the patient fluoroscopically, and the detector is moved until the desired portion of the patient is seen. This delivers a radiation dose to the patient while the patient is being positioned. When using the positioning device, fluoroscopy is not employed until the patient and detector are correctly aligned in the z and x axes. Fluoroscopy is then used only for direct visualization of pathology rather than position.

The positioning device will also alert the operator in the event of patient movement. If the patient moves, differences in output images from the cameras will detect the movement. The patient can be again aligned optically prior to further fluoroscopy. Without the positioning device, patient movement is not detected until misaligned fluoroscopic images are obtained. If a patient event like ingestion, urination or Valsalva are required during imaging, the positioning device allows confirmation of when the event is occurring so that radiation is used only at the appropriate time.

In CT and MM, the positioning device allows visualization of the patient position prior to performing localizer or scanogram scans. The portion of the patient of interest can be chosen optically and only those portions can be localized allowing faster acquisition and decreased dose compared to localizing the entire patient. In ultrasound, the camera will automatically acquire external visualizations of the patient when ultrasound freeze frames are acquired.

As discussed, the systems and methods described herein include many different features, variations and multiple different embodiments. While the positioning device can be built separate to and merely attached to existing medical imaging hardware, the positioning device also may be integrated into a physical structure of the medical imaging devices. Integration into the medical imaging device will allow optimal camera placement, reduce operator and patient interference, allow optimal monitor placement, and improve the device aesthetics. Generally, the optical cameras are positioned adjacent to the detectors in such a way that the camera lenses are included within the same external casing as the medical image detector. This will provide the appearance of a single unit externally. The wiring from the cameras to the monitors will also be encased within the positioning device preventing accidental unplugging, tangling or operator or patient interference. Cleaning of the positioning device will then also occur with regular cleaning of the medical imaging device so as not to require separate cleaning. Monitors can be placed adjacent to the medical imaging monitors such that the operator is able to easily see both the optical images and medical images simultaneously. The monitors can be enclosed by a common encasement also appearing externally as a single unit. Integration of the positioning device, similar to the embodiments described above, can still use information from the medical image device regarding collimation (borders of the medical image) and slice selection. Integration of the positioning device into the medical imaging device can also enable use of a common power source.

The systems and methods have been described in this application in terms of specific embodiments for illustrative purposes and without the intent to limit or suggest that the invention conceived as only one particular embodiment. It is to be understood that the invention is not limited to any single specific embodiments or enumerated variations. Many modifications, variations and other embodiments of the invention will come to mind of those skilled in the art to which this invention pertains, and which are intended to be and are covered by both this disclosure. It is indeed intended that the scope of the invention should be determined by proper interpretation and construction of the claims, including equivalents, as understood by those of skill in the art relying upon the complete disclosure at the time of filing.

In the above Detailed Description, various features may be grouped together to streamline the disclosure. However, the claims may not set forth every feature disclosed herein as embodiments may feature a subset of the features. Further, embodiments may include fewer features than those disclosed in a particular example. Also, although the subject matter has been described in language specific to structural features and/or methodological acts with respect to a particular graphical user interface, it is to be understood that the subject matter defined in the appended claims is not necessarily limited to the specific embodiments, features, or acts described above. Rather, the specific embodiments, features, and acts described above are disclosed as example forms of implementing the claims. Thus, the following claims are hereby incorporated into the Detailed Description, with a claim standing on its own as a separate embodiment.

What is claimed is:

1. A medical imaging system, comprising:
a medical imaging device that obtains medical images of a patient;
a medical imaging monitor that displays the medical images;
at least one camera that tracks a position of the patient during imaging by the medical imaging device; and
a camera monitor that displays the position of the patient as received from the at least one camera,
wherein an output of the at least one camera is correlated with the medical images to illustrate at least one of a position of the patient relative to the medical imaging device and where the medical images are being taken on the patient relative to the medical imaging device.

2. A medical imaging system as in claim 1, wherein the medical imaging device comprises at least one of a computed tomography imaging device, a magnetic resonance imaging device, an ultrasound imaging device, a nuclear medicine imaging device, a mobile radiography imaging device, a stationary radiography imaging device, and a fluoroscopy imaging device.

3. A medical imaging system as in claim 1, wherein the at least one camera outputs crosshairs or a central reticle indicating a mid-position of a field of view of the at least one camera on the camera monitor.

4. A medical imaging system as in claim 1, wherein field of view markers are provided on the camera monitor that indicate an expected field of view of the medical imaging device.

5. A medical imaging system as in claim 4, further comprising a communication device associated with the medical imaging device that creates the field of view markers on an output of the at least one camera to indicate physical scope of medical imaging by the medical imaging device.

6. A medical imaging system as in claim 1, further comprising a recording device that records patient position during imaging by the medical imaging device.

7. A medical imaging system as in claim 1, further comprising shutters placed over a lens of the at least one camera to optionally prevent imaging by the at least one camera.

8. A medical imaging system as in claim 1, further comprising a communication device that sends at least one of medical images of the patient and output of the at least one camera to at least one of a radiology information system and a picture archiving and communication system.

9. A medical imaging system as in claim 1, wherein the output of the at least one camera and medical images are correlated such that an output of the at least one camera is centered and limited to a corresponding field of view of a medical image.

10. A medical imaging system as in claim 9, wherein an output of each camera of the at least one camera is independently centered to the medical image using markers on the camera output.

11. A medical imaging system as in claim 1, wherein the medical imaging device, medical imaging monitor, at least one camera, and camera monitor are integrated into a same physical structure.

12. A method of imaging a patient comprising:
positioning at least one camera with respect to a medical imaging device to obtain views of axes of interest for imaging by the medical imaging device;
imaging a patient with the at least one camera relative to the medical imaging device along the axes of interest of the medical imaging device;
using images from the at least one camera to adjust a position of at least one of the patient and the medical imaging device until an imaging position of the patient is in a field of view of the medical imaging device; and
imaging the patient at the imaging position using the medical imaging device.

13. The method of claim 12, further comprising imaging the patient with the at least one camera during imaging by the medical imaging device, detecting movement of the patient using differences in output images from the at least one camera during the imaging by the medical imaging device, and alerting an operator of the medical imaging device.

14. The method of claim 12, further comprising correlating outputs of the at least one camera with outputs of the medical imaging device whereby the at least one camera is in a known position relative to the medical imaging device such that output images from the at least one camera defines a physical positional relationship of the patient to the medical imaging device.

15. The method of claim 12, further comprising using outputs of the at least one camera to verify that the patient is performing an activity that is to be performed during imaging by the medical imaging device.
